(19) United States
(12) Patent Application Publication
(10) Pub. No.: US 2017/0036016 A1
(43) Pub. Date: Feb. 9, 2017

(54) MAGNETIC CONNECTOR FOR STATIC DISCHARGING DEVICES

(71) Applicant: Desco Industries, Inc., Chino, CA (US)

(72) Inventor: Alejandro Hernandez, Riverside, CA (US)

(21) Appl. No.: 14/820,115

(22) Filed: Aug. 6, 2015

(51) Int. Cl. A61N 1/14; H01R 27/02; H01R 13/62; H01R 9/05

(52) U.S. Cl. CPC A61N 1/14; H01R 9/05; H01R 27/02; H01R 13/6205

(57) ABSTRACT

A magnetic connecting device for closing an electrical circuit through the use of magnetic force and contact plates. The contact plates are configured and shaped in a manner which allows for swiveling without disconnecting.

### BACKGROUND

The present invention relates generally to electrically conductive body bands (e.g., wrist bands, ankle bands, and the like) used to drain static electricity from the human body.

During the handling of static-sensitive electronics, static electricity caused by the handler may damage certain components of the electronics. This can be a major problem for an electronics manufacturer which require workers to constantly handle the electronics. In order to minimize or avoid damage to electronics through static electricity, manufacturers drain or discharge static electricity from the workers by attaching them to groundings cords. One method manufacturers attach workers to grounding cords is by having workers wear an electrically conductive body band which is attached to a grounding cord. It is generally preferable that the grounding cord is releasable from the body bands such that a worker can easily disconnect and reconnect themselves from the grounding cord when they wish to ambulate away from the work area and come back.

Two popular methods of creating a releasable connection between a wristband and the grounding cord is through a magnetic connector or a snap connector, which uses a spring-loaded and/or frictionally engaged connectors. Each has their own benefit and drawbacks.

The magnetic connector has the benefit of being easily removable and causing less wear. A magnetic connector attaches the wristband and grounding cord through magnetic force. These connectors usually incorporate a single magnet which is made of conductive material or covered with a conductive metal to transfer static electricity from the wristband to the grounding cord.

However, in this age of high-tech microelectronics, the same large factory may have a substantial group of employees (workers) who use dual-path constant-monitoring electrostatic grounding devices and elements, and another large group of employees who use single-path electrostatic grounding devices and elements. The dual-path devices are for the more sensitive products, and the worker's grounding circuits are monitored to assure that no undetected failure in the grounding circuit occurs. However, because current dual-path connectors that uses magnetic force requires two magnets, one for each connection, the wristbands for single-path connections are incompatible with dual path ground connectors. Furthermore, because there are two connection points, the ground wire is unable to have 360 degree swivel, making its use cumbersome for the worker.

Electrostatic grounding devices that can switch between dual path or single path is highly desirable. This allows one or more employees to immediately change from a dual path portion of a factory to a single path portion and vice versa without having to change body bands. This feature thus saves time and promotes maximum use of equipment by having a single device serve multiple functions. One solution to this problem has been the use of snap connectors. However, a major problem with snap connectors is that they use spring force and friction to maintain a connection. These connections, therefore, wear out overtime. When the connection of a snap connector becomes loose, the wristband will easily detach from the grounding cord, thus rendering the electrostatic grounding device inoperable.

Therefore, it would be desirable for a connecting device that is less susceptible to wear and allows for 360 degree swivel. The present invention solves this and other needs.

### SUMMARY OF THE INVENTION

In the most general aspect, the invention includes an apparatus for closing one or more electrical circuits through contact between two contact surfaces and/or contact between a contact surface and a contact pin, wherein the contacts create a separable connection that is maintained by magnetic force. In one aspect, the contact surfaces are configured in a manner such that the connection between the contact surfaces can freely swivel at least 360 degrees. In another aspect the connection can freely swivel continuously. In yet another aspect, the contact surfaces are configured to allow for swivel but prevent disconnection through a sheering force.

In one aspect, the invention includes a magnetic connecting device comprising: a first connector with a coaxial contact point comprising a first contact surface that is covering a magnet and is annularly shaped; and a second contact surface surrounded by the first contact surface and separate from the first contact surface forming a recess between the first contact surface and second contact surface.

In another aspect, the present invention includes a magnetic connecting device of comprising a second connector with a third contact surface that is annularly shaped, configured to closely mate with the first connector, and is held in place by the magnet when the second connector is connected with the first connector.

In yet another aspect, the present invention includes a magnetic connecting device wherein the first connector forms an annular lip that surrounds the third contact surface when the first connector is connected with the second connector.

In still another aspect, the present invention includes a magnetic connecting device wherein the third contact surface has an annular shaped protrusion that fits within the recess and is in contact with the first contact surface but not the second contact surface when the first connector is connected to the second connector.

In another aspect, the present invention includes a magnetic connecting device wherein the second connector further comprises a contact pin.

In yet another aspect, the present invention includes a magnetic connecting device wherein the contact pin is located in the center of an aperture created by the third contact surface.

In yet another aspect, the present invention includes a magnetic connecting device wherein the contact pin is spring loaded.

In yet another aspect, the present invention includes a magnetic connecting device wherein the contact pin contacts the second contact surface when the first connector is connected to the second connector.

In yet another aspect, the present invention includes a magnetic connecting device wherein the second contact surface depresses the contact pin when the first connector is connected to the second connector.

In still another aspect, the present invention includes a magnetic connecting device wherein the first connector is attached to a body band.

In another aspect, the present invention includes a magnetic connecting device wherein the body band is a wristband.

In another aspect, the present invention includes a magnetic connecting device wherein the second connector comprises a coaxial wire with a first conducting wire within the coaxial wire is connected to the contact pin and a second conducting wire connected to the third contact surface.

In another aspect, the present invention includes a magnetic connecting device wherein the first connector and second connector are configured to have 360 degree swivel when connected.

In yet another aspect, the present invention includes a magnetic connecting device wherein the magnet exerts four pounds of magnetic force connecting the first connector to the second connector, when connected.

In another aspect, the present invention includes a magnetic connecting device wherein the contact pin is gold plated.

In another aspect, the present invention includes a magnetic connecting device wherein the third contact surface is made of ledloy.

In another aspect, the present invention includes a magnetic connecting device wherein the coaxial wire is coiled to allow for stretching.

In another aspect, the present invention includes a magnetic connecting device wherein the wristband comprises an inner surface that is electrically conductive.

In another aspect, the present invention includes a magnetic connecting device comprising: a first connector with a coaxial contact point comprising: a first contact surface that is covering a magnet and is annularly shaped; a second contact surface surrounded by the first contact surface and separate from the first contact surface forming a recess between the first contact surface and second contact surface; and a second connector with a third contact surface that is annularly shaped, configured to closely mate with the first connector, and is held in place by the magnet when the second connector is connected with the first connector.

In another aspect, the present invention includes a magnetic connecting device comprising: a first connector with a coaxial contact point comprising: a first contact surface that is covering a magnet and is annularly shaped; a second contact surface surrounded by the first contact surface and separate from the first contact surface forming a recess between the first contact surface and second contact surface; a second connector comprising: a third contact surface that is annularly shaped, configured to closely mate with the first connector, and is held in place by the magnet when the second connector is connected with the first connector; and a spring loaded contact pin that is located in the center of an aperture created by the third contact surface.

### BRIEF DESCRIPTION OF THE DRAWINGS

FIG. 1 illustrates an expanded view an exemplary magnetic connector.

FIG. 2 illustrates a cross sectional view of the magnetic connector in FIG. 1

FIG. 3A illustrates the magnetic connector of FIG. 1 connected to a wrist band and coiled wire.

FIG. 3B illustrates the magnetic connector of FIG. 1 disengaged with a wrist band and coiled wire.

### DETAILED DESCRIPTION OF THE PREFERRED EMBODIMENTS

As will be described hereinafter in greater detail, the various embodiments of the present invention relate to electric static grounding devices. More specifically, the present invention relates to a swiveling magnetic connector for connecting an electrostatic body band to a grounding device. For purposes of explanation, specific nomenclature is set forth to provide a thorough understanding of the present invention. Description of specific applications and methods are provided only as examples. Various modifications to the embodiments will be readily apparent to those skilled in the art and the general principles defined herein may be applied to other embodiments and applications without departing from the spirit and scope of the invention. Thus the present invention is not intended to be limited to the embodiments shown, but is to be accorded the widest scope consistent with the principles and steps disclosed herein.

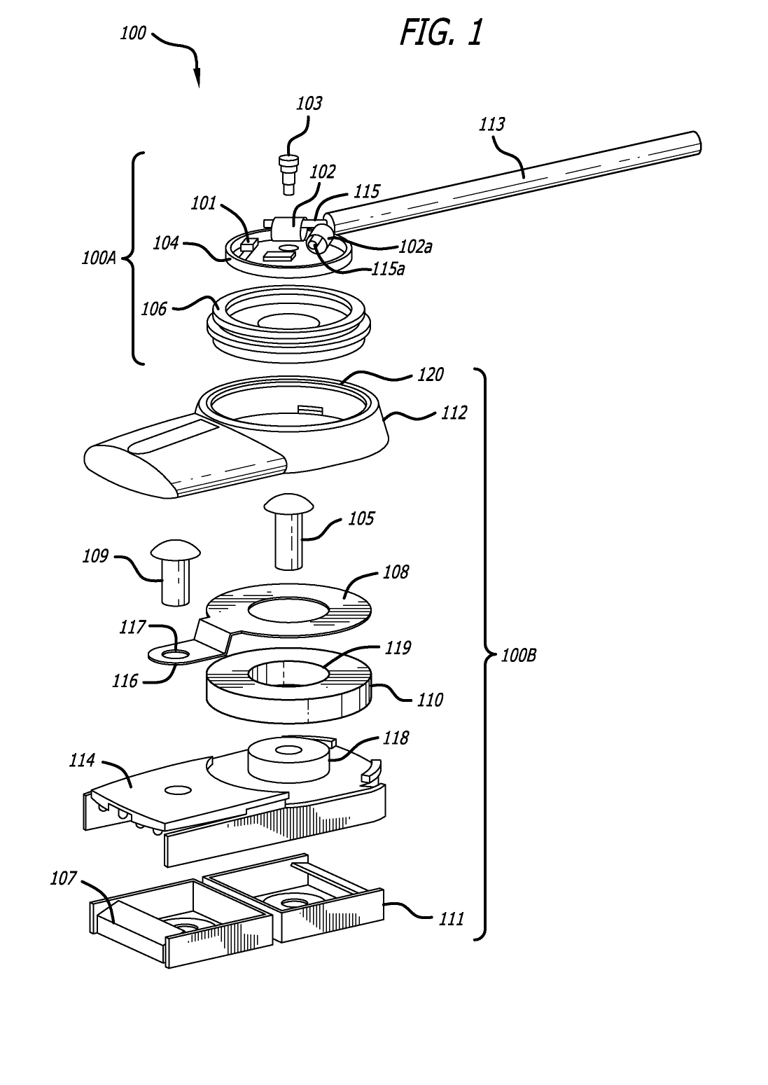

FIG. 1 illustrates an expanded view an exemplary magnetic connector 100 used for a grounding device according to one embodiment. In one embodiment, magnetic connector may have a ground connector assembly 100A that may magnetically attach to socket 100B. Socket 100B may have a coaxial contact surface creating a single contact point for allowing a single or coaxial ground connector assembly 100A to contact when engaged with socket 100B. Engaged, as discussed herein, is defined as when corresponding flanges and recesses between objects are aligned and connected. The coaxial contact eliminates the needs for physically separate contact surfaces and wiring. Instead, a coaxial cable may be used without forking the wiring. This prevents wire tangle when ground connector assembly 100A swivels on socket 100B. Additionally, it will be apparent to one skilled in the art that coaxial contacts allow socket 100B to be compatible with a single channel versions of ground connector assembly 100A.

In one embodiment, ground connector assembly 100A may include a ground cord 113. Ground cord 113 may be dual path insulated wire or a coaxial cable with two separately insulated electrically conducting wires 115 and 115a. In one embodiment wires 115 and 115a may be tinsel wire. Tinsel wire is useful in this application because it allows for mechanical flexibility while resisting metal fatigue. In alternative embodiments ground cord 113 may be a single path wire cable. However, a dual path ground cable has the benefit of a redundant ground path for electricity to travel in case one of the ground connections fail. Furthermore, some static electricity monitoring devices only function with dual path connections.

According to one embedment, wires 115 and 115a may be connected to a crimp splice 102 and 102a respectively. Crimp splices 102 and 102a may be made of any electrically conductive material. Crimp splices 102 and 102a may be attached to a printed circuit board 104. Printed circuit board 104 may contain circuitry which allows electrical current flow from the wires 115 and 115a to contact pin 103 and outer contact ring 106 respectively while maintaining an open circuit between each wires 115 and 115a. In one embodiment, there may be one or more resistors 101 and 101a attached to printed circuit board 104 to limit electrical current flow.

Contact pin 103 may be made of an electrically conductive material which connects to printed circuit board 104. Printed circuit board 104 may have circuitry that connects contact pin 103 to wire 115 through crimp splice 102. Printed circuit board 104 may have an aperture in the center which contact pin 103 penetrates. Contact pin 103 may be gold plated to aid in conducting electricity.

Printed circuit board 104 may be mounted on or into ring shaped contact 106. Ring shaped contact 106 maybe be made of one or more electrically conductive materials that are also attracted by a magnet. In one embodiment, Ring shaped contact 106 may be made from a chrome plated ledloy. In an alternative embodiment, wires and resistors may replace the function of the printer circuit board. In yet another alternative embodiment, ground connector assembly 100A may be made without contact pin 108 so that ground connector assembly is a single path ground connector.

In one embodiment, socket 100B may be configured for receiving ground connector assembly 100A and maintaining an electrical conductive connection to dual conductor wire 113 through contact with contact pin 103 and outer contact ring 106. Socket 100B may be configured or shaped to closely mate with ground connector assembly 100A while still allowing for 360 degree swivel. Socket 100B may also be compatible with a single path version of ground connector 100A where ground connector 100A lacks a contact pin. In this manner, socket 100B may be used for dual or single path grounding devices.

According to one embodiment, socket 100B may have an annular shaped metal contact plate 108 for connecting to and maintaining an electrical conductive connection with outer contact ring 106. Contact plate 108 may have a circular center aperture for allowing rivet 105 to pass through without contacting contact plate 108.

Rivet 105 may be made of an electrically conductive material that protrudes in such a manner that it may abut contact pin 103 when socket 100B receives or connects to ground connector assembly 100A. In one embodiment, contact pin 103 may be spring loaded, and rivet 105 may protrude in a manner such that rivet 105 forces contact pin 103 to retract and press against rivet 105 when socket 100B receives or connects to ground connector assembly 100A. The spring loading aids contact pin 103 in maintaining a connection with rivet 105 even if the contact pin 103 wears or is slightly damaged.

Contact plate 108 may have an appendage 116 with a eyelet 117 for rivet 109 to pass through and secure contact plate 108 to an electrically insulating body 114. Rivet 109 may also be made of an electrically conductive material and maintain a short circuit connection with contact plate 108.

In one embodiment, a ring shaped magnet 110 may be sandwiched between the annular portion of contact plate 108 and body 114. Body 114 may be made of a electrically insulated material such as acrylonitrile butadiene styrene (ABS). Body 114 may have a cylindrical protrusion or flange 118 that is shaped and/or configured to receive magnet 110 and fit snuggly within magnet 110's opening 119. In alternative embodiments, flange 118 and corresponding opening 119 may be of different shapes, such as a square, rectangle, star, or any other shape. The flange 118 may also have a lumen for rivet 105 to penetrate through.

Socket 100B may have two back plates 107 and 111 mounted underneath body 114. Back plates 107 and 111 may be secured to body 114 by rivets 109 and 105 respectively. In an alternative embodiment, back plates 107 and 111 may be secured to body 114 in other manners. Back plates 107 and 111 may also be conductively attacked to rivets 109 and 105 respectively. Body 114 may insulate and separate back plates 107 and 111 from electrically shorting. Back plates 107 and 111 may be configured to connect to a body band for wicking static electricity from the worker's body through the dual path ground connection.

Socket 100B may have a cap 112 that is made of electrically insulating material such as ABS. Cap 112 may be configured or molded to secure onto body 114. In one embodiment, cap 112 may aid in securing contact plate 108 and magnetic ring 110 to body 114. Cap 112 may have a circular opening that forms a annular lip 120 above and around contact plate 108. Annular lip 120 may be configured to surround and prevent outer contact ring 106 from sliding off contact plate 108 when the ground connector assembly 100A is engaged with socket 100B.

In one embodiment, rivet 105 and contact plate 108 may create an annular recess within the opening of contact plate 108 between rivet 105 and contact plate 108. The recess may be configured to receive a ring-shaped protrusion or flange on outer contact ring 106 (not shown) to help prevent contact ring 106 from sliding off contact plate 108, when connected. The ring-shaped protrusion or flange on outer contact ring 106 may only maintain an electrical connection to contact plate 108 and not rivet 105 when ground connector assembly 100A is engaged with socket 100B.

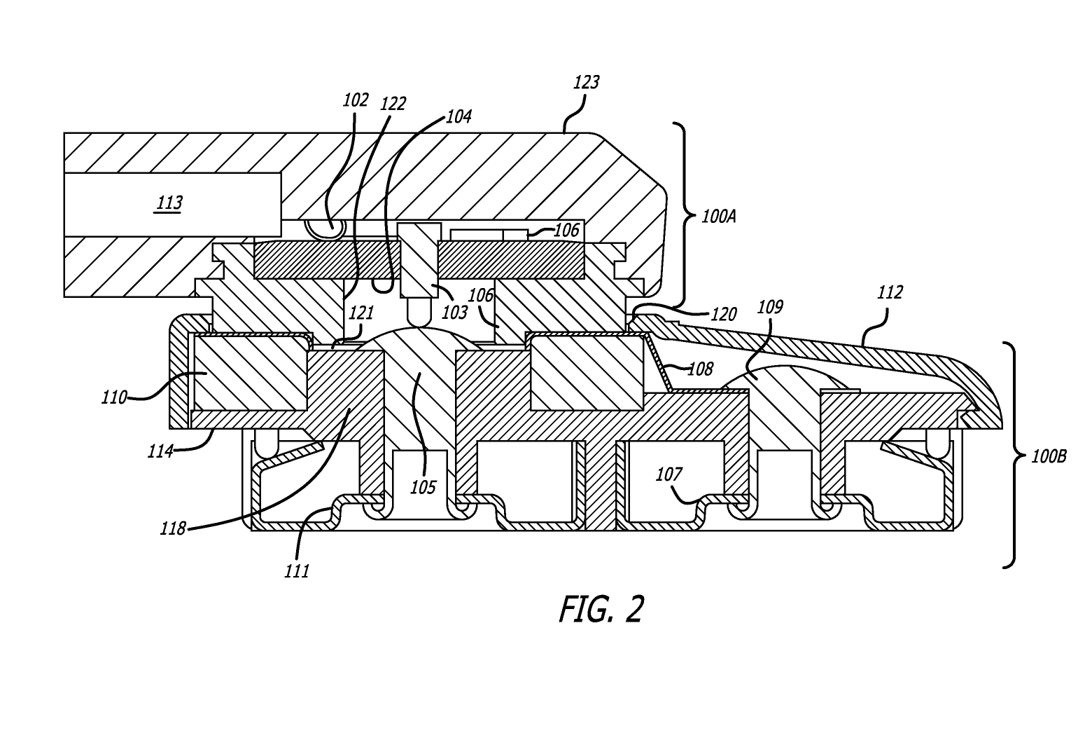

FIG. 2 is a cross section view of exemplary magnetic connector 100 used for a grounding device where ground connector assembly 100A is engaged with socket 100B. Socket 100B includes a ring shaped magnet 110 that is seated in a body 114 wherein the center opening of magnet 110 surrounds flange 118, which is a cylindrical protrusion of body 114. Buckle cap 112 and contact plate 108 may aid in securing magnet 110 onto body 114. Rivet 109 may aid in securing contact plate 108 and back plate 107 onto body 114. Rivet 29 may also create an electrical short between back plate 107 and contact plate 108.

Rivet 105 may penetrate through the center of flange 118 of body 214. Rivet 105 may secure back plate 111 onto body 114 and create an electrical short with back plate 111.

A contact ring 106 may be removeably connected to contact plate 108 by the magnetic force of magnet 110. Contact ring 106 may by cylindrically shaped such that, when connect to contact plate 108, the annular shaped recess created by the annular lip 120 of buckle cap 112 surrounds at least a portion of the outer circumference of contact ring 106. Contact plate 108 and annular lip 120 may be configured in a ring shape manner such that ground connector assembly 100A may swivel easily while engaged with socket 100B yet prevent contact plate 108 from slipping off of contact ring 106. In one embodiment, magnet 110 will exert between three to six pounds of force connecting socket 100B to connector assembly 100A. In the most preferred embodiment, magnet 110 will exert approximately four pounds of force connecting socket 100B to connector assembly 100A when the two are engaged. In an alternative embodiment the magnetic connector may utilize two magnets, one on the socket and the other on the connector assembly, to create the required amount of attachment force. However, a single magnet is preferred because a single larger magnet may be used to create the attachment force allowing for a larger contacting surface area. The larger contacting surface area aids in preventing the connector assembly and the socket from slipping off when engaged. Furthermore, a larger surface area allows for larger and/or multiple flanges and corresponding recesses between the connector assembly and socket to prevent slippage while maintaining the preferred magnetic strength. A yet additional benefit of a single magnet is that a user will not need to deal with matching magnet polarities between the socket and the connector assembly.

In one embodiment, flange 118 of body 114 may be configured to create a annular recess 121 between contact plate 108 and rivet 105. Contact ring 106 may have a protrusion or flange 122 to fit within the recess between contact plate 108 and rivet 105 but remain unconnected from rivet 105 when ground connector assembly 100A and socket 100B are engaged.

Contact ring 106 may be conductively connected to a grounding wire from dual channel wire 113 (not shown) through PCB 114 and a resistor 101a in series. A contact pin 103 may be connected to a second grounding wire of dual channel connector 113 (not shown) through circuitry on PCB 104 in a manner that is insulated from contact ring 106 (not shown). In one embodiment, a second resister may be close the electrical connection between PCB 114 and the second grounding wire (not shown).

In one embodiment, contact pin 103 may be spring loaded such that the tip of contact pin 103 depresses. Contact pin 103 may be configured and/or situated on PCB 104 such that when contact ring 106 is properly fitted within the annulus formed by cap 112 and recess 121 and in contact with contact plate 108, rivet 105 depresses contact pin 103.

Ground connector assembly 100A may have a cap 123. Cap 123 may be configured to house and help secure all components of connector assembly 100A.

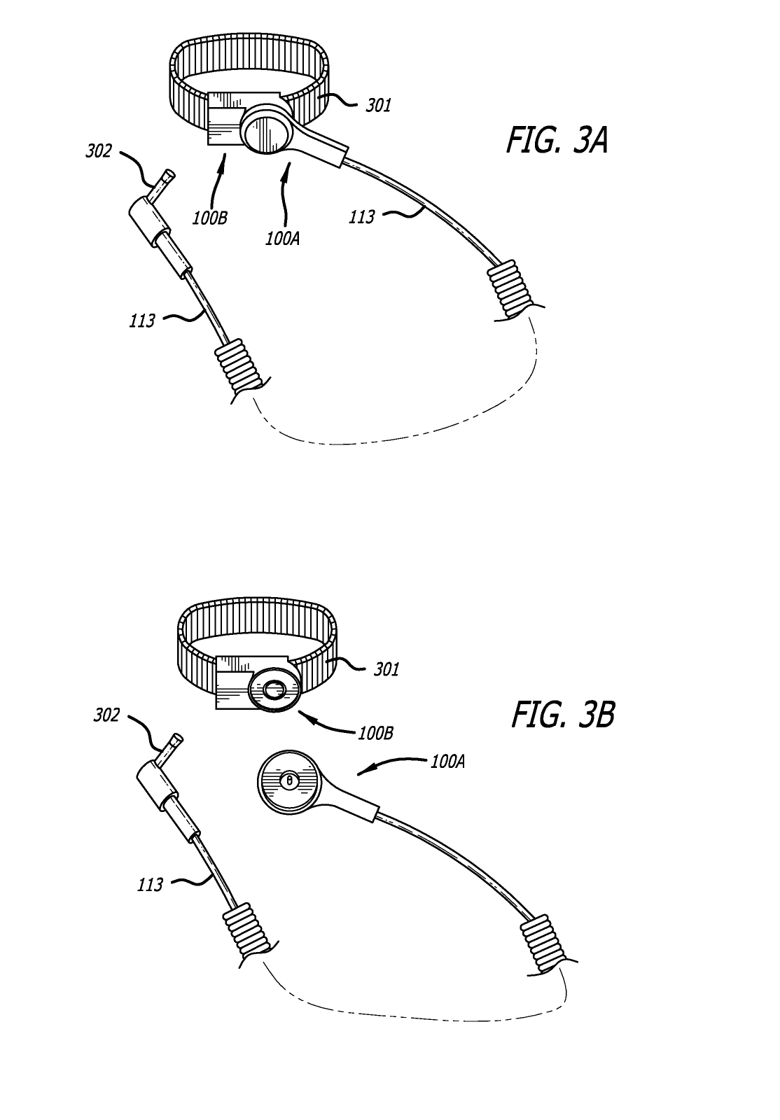

FIG. 3A and FIG. 3B illustrates magnetic connector 100 of FIG. 1 with a wrist strap 301 connected to socket 100B. FIG. 3A depicts connector 100 with ground connector assembly 100A engaged with socket 100B. FIG. 3B depicts connector 100 with ground connector assembly 100A disengaged from socket 100B. In one embodiment, the inner portion of wristrap 301 may be made of a conductive material that attaches to back plate 107 and 111 of socket 100B. Wristrap 301 may be made in a manner such that it may expand and stretch to allow a user to easily put on and take off the wrist strap. Wrist strap 301 may also have a insulated covering preventing a user from accidentally shorting circuitry with wrist strap 301.

In one embodiment, dual conductor wire 113 may have coils to allow wire 113 to stretch and retract. Dual conductor wire 113 may terminate with a coaxial plug 302. In an alternative embodiment, wire 113 may be a single path wire connector with a single path plug.

While particular embodiments of the present invention have been described, it is understood that various different modifications within the scope and spirit of the invention are possible. The invention is limited only by the scope of the appended claims

I claim:

1. A magnetic connecting device comprising:
a first connector with a coaxial contact point comprising a first contact surface that is covering a magnet and is annularly shaped; and
a second contact surface surrounded by the first contact surface and separate from the first contact surface forming a recess between the first contact surface and second contact surface.

2. The magnetic connecting device of claim 1 further comprising a second connector with a third contact surface that is annularly shaped, configured to closely mate with the first connector, and is held in place by the magnet when the second connector is connected with the first connector.

3. The magnetic connecting device of claim 2 wherein the first connector forms an annular lip that surrounds the third contact surface when the first connector is connected with the second connector.

4. The magnetic connecting device of claim 3 wherein the third contact surface has an annular shaped protrusion that fits within the recess and is in contact with the first contact surface but not the second contact surface when the first connector is connected to the second connector.

5. The magnetic connecting device of claim 4 wherein the second connector further comprises a contact pin.

6. The magnetic connecting device of claim 5 wherein the contact pin is located in the center of an aperture created by the third contact surface.

7. The magnetic connecting device of claim 6 wherein the contact pin is spring loaded.

8. The magnetic connecting device of claim 7 wherein the contact pin contacts the second contact surface when the first connector is connected to the second connector.

9. The magnetic connecting device of claim 8 wherein the second contact surface depresses the contact pin when the first connector is connected to the second connector.

10. The magnetic connecting device of claim 9 wherein the first connector is attached to a body band.

11. The magnetic connecting device of claim 10 wherein the body band is a wristband.

12. The magnetic connecting device of claim 11 wherein the second connector comprises a coaxial wire with a first conducting wire within the coaxial wire is connected to the contact pin and a second conducting wire connected to the third contact surface.

13. The magnetic connecting device of claim 12 wherein the first connector and second connector are configured to have 360 degree swivel when connected.

14. The magnetic connecting device of claim 13 wherein the magnet exerts four pounds of magnetic force connecting the first connector to the second connector, when connected.

15. The magnetic connecting device of claim 14 wherein the contact pin is gold plated.

16. The magnetic connecting device of claim 15 wherein the third contact surface is made of ledloy.

17. The magnetic connecting device of claim 16 wherein the coaxial wire is coiled to allow for stretching.

18. The magnetic connecting device of claim 12 wherein the wristband comprises an inner surface that is electrically conductive.

19. A magnetic connecting device comprising:
a first connector with a coaxial contact point comprising:
a first contact surface that is covering a magnet and is annularly shaped;
a second contact surface surrounded by the first contact surface and separate from the first contact surface forming a recess between the first contact surface and second contact surface; and
a second connector with a third contact surface that is annularly shaped, configured to closely mate with the first connector, and is held in place by the magnet when the second connector is connected with the first connector.

20. A magnetic connecting device comprising:
a first connector with a coaxial contact point comprising:
a first contact surface that is covering a magnet and is annularly shaped;
a second contact surface surrounded by the first contact surface and separate from the first contact surface forming a recess between the first contact surface and second contact surface;
a second connector comprising:
a third contact surface that is annularly shaped, configured to closely mate with the first connector, and is held in place by the magnet when the second connector is connected with the first connector; and
a spring loaded contact pin that is located in the center of an aperture created by the third contact surface.
